(19) United States
(12) Patent Application Publication
(10) Pub. No.: US 2004/0122476 A1
(43) Pub. Date: Jun. 24, 2004

(54) Emergency medical devices with multiple displays

(76) Inventor: Peter Wung, Redmond, WA (US)

Correspondence Address: SHUMAKER & SIEFFERT, P. A., 8425 SEASONS PARKWAY, SUITE 105, ST. PAUL, MN, 55125, US

(21) Appl. No.: 10/712,597

(22) Filed: Nov. 13, 2003

Related U.S. Application Data
(60) Provisional application No. 60/436,421, filed on Dec. 24, 2002.

(51) Int. Cl. A61N 1/39; A61B 5/04

(52) U.S. Cl. 607/5; 600/525; 600/523

(57) ABSTRACT

In general, the invention is directed to an emergency medical device with multiple display monitors for displaying patient parameters to an operator of the medical device. In particular, the display monitors of the emergency medical device are arranged such that the operator may continuously view the patient parameters in order to constantly monitor the condition of a patient. The display monitors may, for instance, be arranged to allow the operator of the medical device to view measured patient parameters while a cover of the medical device is closed or during transportation of the medical device and the patient. For example, the medical device may include a secondary display monitor on the same side as a handle for viewing patient parameters during transportation of the patient and the medical device. The display monitors may each display the same patient parameters, a portion of the same patient parameters, or completely different patient parameters to the operator.

This application claims priority from U.S. Provisional Application Serial No. 60/436,421, filed Dec. 24, 2002, the entire content of which is incorporated herein by reference.

### TECHNICAL FIELD

The invention relates to medical devices, and more particularly, to medical devices for monitoring or treating cardiac emergencies.

### BACKGROUND

Cardiac arrest is a life-threatening medical condition that may be treated with external defibrillation. External defibrillation includes applying electrodes to a chest of a patient and delivering an electric shock to the patient to depolarize a heart of the patient and restore normal sinus rhythm. The chances that the heart of the patient can be successfully defibrillated increase significantly if a defibrillation pulse is applied quickly. Typically, a paramedic, an emergency medical technician, or other person trained in defibrillation techniques, uses an external defibrillator to monitor and assess the condition of a patient, and, if necessary, to defibrillate the heart of the patient. The external defibrillator is coupled to electrodes that are attached to the patient for monitoring of the patient and delivery of electric shocks to the patient.

External defibrillators include a display monitor to display patient parameters to an operator of the device. However, where the defibrillator includes a cover, the display monitor is usually located on a side of a cover of the defibrillator that is not viewable when the cover is closed. Even where the defibrillator does not include a cover, the display monitor is usually located on a side of the defibrillator that cannot be seen by the operator while the defibrillator is being carried by a handle, e.g., a side of the defibrillator that faces away from the operator while the operator holds the defibrillator by the handle. Thus, the operator of the external defibrillator may be unable to view the patient parameters displayed by display monitor of the defibrillator during transport or when the cover is closed. Therefore, there may be a brief period of time in which the operator does not monitor the condition of the patient, which may be unsafe for the patient.

### SUMMARY

In general, the invention is directed to an emergency medical device with multiple display monitors for displaying patient parameters to an operator of the medical device. In particular, display monitors of the emergency medical device are arranged such that the operator may continuously view the patient parameters in order to constantly monitor the condition of a patient. The display monitors may be arranged to allow the operator of the medical device to view measured patient parameters when the emergency medical device or portions of the device occupy different orientations relative to the operator, e.g., while a cover of the medical device is closed or during transportation of the medical device and the patient. The emergency medical device may comprise any external therapeutic, monitoring or diagnostic emergency medical device. However, for exemplary purposes, the invention will be described in the context of external defibrillators.

As described above, an emergency medical device, such as an external defibrillator, includes two or more display monitors that are arranged to allow the operator of the defibrillator to continuously view measured patient parameters. As an example, the defibrillator may include a first display monitor that is located on the “front” surface of the defibrillator, i.e., on a surface of the defibrillator that is not visible when the defibrillator is being transported, e.g., being carried by its handle, and a second display monitor that is located on the same surface on which a handle of the defibrillator is located, i.e., a “top” surface of the defibrillator that is visible when the defibrillator is being carried by the handle. In this case, the defibrillator operator may view the patient parameters on the second display monitor while the operator is transporting the patient and carrying the defibrillator.

In another example, the external defibrillator may include a cover that has the first and second display monitors located on opposite sides of the cover, thereby allowing the operator to view the patient parameters while the cover of the external defibrillator is closed. In yet another example, the external defibrillator may include a first display monitor integrated within a housing or cover of the external defibrillator and be electrically coupled to a second display monitor via a port so that the external defibrillator drives the second display monitor. In this manner, the second display monitor is detached from the external defibrillator, but may display patient parameters to the operator during transport or while the cover of the external defibrillator is closed.

The first and second display monitors of the external defibrillator may display patient parameters to the operator concurrently or successively. For example, the first monitor may display a first set of patient parameters to the operator while a cover of the external defibrillator is closed and be shut off when the cover is opened, and the second display monitor may display a second set of patient parameters to the operator when the cover is opened. The display monitors may each display the same patient parameters, a portion of the same patient parameters, or completely different patient parameters to the operator. For example, one of the display monitors may display only a subset of the patient parameters that the other display monitor displays. The first display monitor may, for instance, display a heart rate, an ECG waveform, patient information such as age, weight, and the like, as well as external defibrillator status features such as a battery icon and a charging icon. The second display monitor may only display the vital patient parameters such as the heart rate and ECG of the patient. Accordingly, the content and format of the information displayed by the monitors may vary.

The defibrillator operator may interact with the external defibrillator via an input medium, such as a keypad, touch screen or a peripheral pointing device, in order to identify patient parameters for each of the display monitors to display. In this manner, the defibrillator operator may configure each of the display monitors of the defibrillator to display the same patient parameters, a portion of the same patient parameters or completely different patient parameters.

In one embodiment, the invention provides an emergency medical device comprising at least one sensor to measure one or more patient parameters, a first display monitor to display at least a first subset of the patient parameters to an operator, and a second display monitor to display at least a second subset of the patient parameters to the operator.

In another embodiment, the invention provides a method comprising measuring one or more patient parameters, displaying a first subset of the measured patient parameters to an operator via a first display monitor of an emergency medical device, and displaying a second subset of the measured patient parameters to the operator via a second display monitor of the emergency medical device.

The invention may provide a number of advantages. In general, the invention allows an operator of an emergency medical device to continuously view measured patient parameters to constantly monitor the condition of the patient, without regard to changes in orientation of the emergency medical device relative or the viewing perspective of the operator. In particular, the emergency medical device includes multiple display monitors to allow the operator of the medical device to view measured patient parameters when, for example, the cover is closed or the medical device is being carried by its handle during transport. In this manner, the safety of the patient is not jeopardized due to the inability of the operator to view measured patient parameters.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram showing a patient coupled to an external defibrillator.

FIG. 2 is a block diagram illustrating exemplary components of the external defibrillator of FIG. 1 in further detail.

FIG. 3 is a schematic diagram illustrating an exemplary external defibrillator that includes multiple display monitors.

FIGS. 4A and 4B are schematic diagrams illustrating an external defibrillator that includes multiple display monitors integrated within a cover.

FIGS. 5A and 5B are schematic diagrams illustrating an exemplary external defibrillator that includes at least one of the multiple display monitors is detached from the external defibrillator.

FIG. 6 is a flow diagram illustrating exemplary operation of an external defibrillator with multiple display monitors displaying patient parameters to an operator in accordance with the invention.

FIG. 7 is a flow diagram illustrating exemplary operation of an external defibrillator that switches between display monitors upon opening or closing of a cover of the external defibrillator.

### DETAILED DESCRIPTION

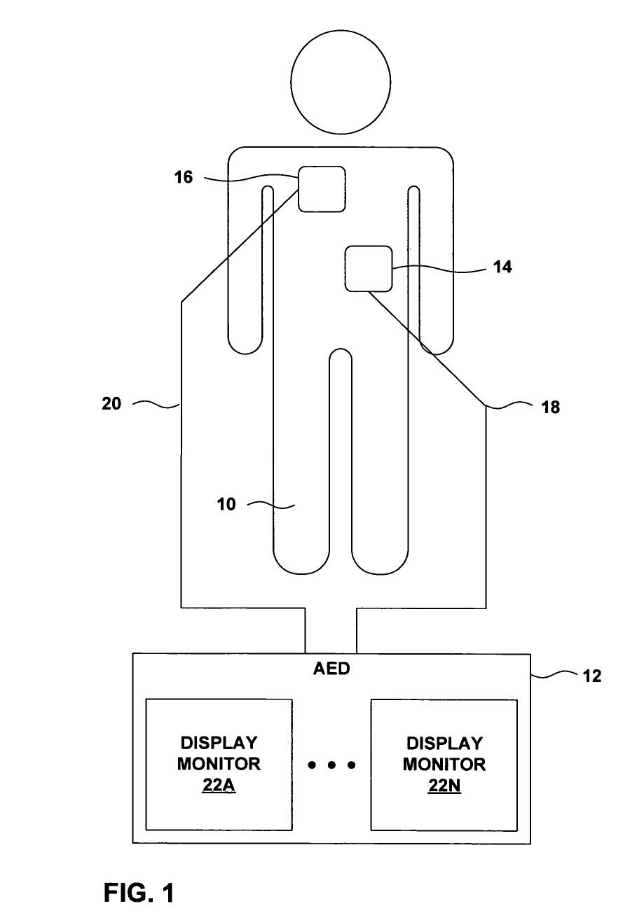

FIG. 1 is a block diagram showing a patient 10 coupled to an emergency medical device, such as an external defibrillator 12. Although the invention may be applicable to a variety of external emergency medical devices, various embodiments will be described herein in the context of external defibrillators for exemplary purposes. External defibrillator 12 may take the form of, for example, a defibrillator/monitor or an automatic external defibrillator (AED).

External defibrillator 12 administers defibrillation therapy to patient 10 via electrodes 14 and 16, which may be hand-held electrode paddles or adhesive electrode pads placed on the skin of patient 10. The body of patient 10 provides an electrical path between electrodes 14 and 16. Electrodes 14 and 16 are coupled to defibrillator 12 via conductors 18 and 20 and a stimulation interface (not shown). In a typical application, the stimulation interface includes receptacles and conductors 18 and 20 plug into one or more designated receptacles. External defibrillator 12 may sense electrical impulses or signals via electrodes 14 and 16. In this manner, defibrillator 12 may measure one or more patient parameters such as heart rate, electrocardiogram signals (ECGs), and the like. Defibrillator 12 may also be coupled to an array of dedicated sensing electrodes (not shown), such as an array of ten electrodes arranged in the well-known configuration for collection of a twelve-lead ECG, and to other sensors, such as a pulse-oximeter or blood pressure cuff. Electrical impulses or signals, e.g., defibrillation shocks, may also be delivered from defibrillator 12 to patient 10 via electrodes 14 and 16.

External defibrillator 12 includes display monitors 22A-22N (“display monitors 22”) that display respective sets of patient parameters to an operator. In accordance with the invention, display monitors 22 are arranged to allow the operator of defibrillator 12 to continuously view patient parameters for constant monitoring of the condition of patient 10. In one example, display monitors 22 are arranged to allow the operator of external defibrillator 12 to view patient parameters during transportation of patient 10. For instance, a major surface of one of display monitors 22 is located the same side of external defibrillator 12 as a handle, such that the operator may view the respective display monitor while carrying external defibrillator 12 by the handle.

In another example, display monitors 22 are arranged to allow the operator to view patient parameters whether a cover of external defibrillator 12 is open or closed. For instance, display monitors 22 may be located on opposite sides of the cover. Specifically, a first one of display monitors 22 is located on an outside surface of the cover of external defibrillator 12 and another one of display monitors 22 is located on an inside surface of the cover of external defibrillator 12. In this manner, external defibrillator 12 offers the advantage that external defibrillator 12 may be carried or have its cover closed and certain important data may still be viewable.

In some embodiments, display monitors 22 of external defibrillator 12 may be integral to the structure of external defibrillator 12. For instance, in the examples described above, display monitors 22 are integrated within either a housing or a cover of external defibrillator 12. Alternatively, defibrillator 12 may be electrically coupled to and drive at least one of display monitors 22 via a video or other display port. For example, defibrillator 12 may include a main display that is integral part of defibrillator 12 and electrically couple to a second display that is detached from defibrillator 12. The second display may be coupled to external defibrillator 12 via either a wireless connection, e.g., 802.11a, Bluetooth, or IRDA, or a wired port and a connector. In some embodiments, the second display monitor 22 may be a freestanding monitor, while in other embodiments the second display is a component of another device, such as a laptop or handheld computer, a personal digital assistant (PDA), a cellular telephone, or a wristwatch.

Each of display monitors 22 of the external defibrillator 12 may display patient parameters to the operator concurrently or successively. For example, display monitor 22A may display a first set of patient parameters to the operator while a cover of the external defibrillator 12 is closed and shut off when the cover is opened, and display monitor 22B may display a second set of patient parameters to the operator when the cover is opened. Display monitors 22 may each display the same patient parameters, a portion of the same patient parameters, or completely different patient parameters to the operator. For example, a main display monitor may display a detailed set of patient parameters, and a different display monitor that is viewable during transportation of the patient may only display a subset of the patient parameters displayed by the main display monitor, e.g., the most critical of the patient parameters. Accordingly, the content and format of the information displayed by the monitors may vary.

Display monitors 22 may comprise, for example, Cathode Ray Tube (CRT) display monitors, Light Emitting Diode (LED) monitors, Liquid Crystal Display (LCD) monitors, or plasma display monitors. As described above, one of the display monitors 22 could be a freestanding monitor or a component of another device, such as a laptop or handheld computer, a personal digital assistant (PDA), a cellular telephone, or a wristwatch. Further, although described in terms of defibrillators for exemplary purposes, the emergency medical device may comprise an external therapeutic emergency medical device or an external diagnostic emergency medical device.

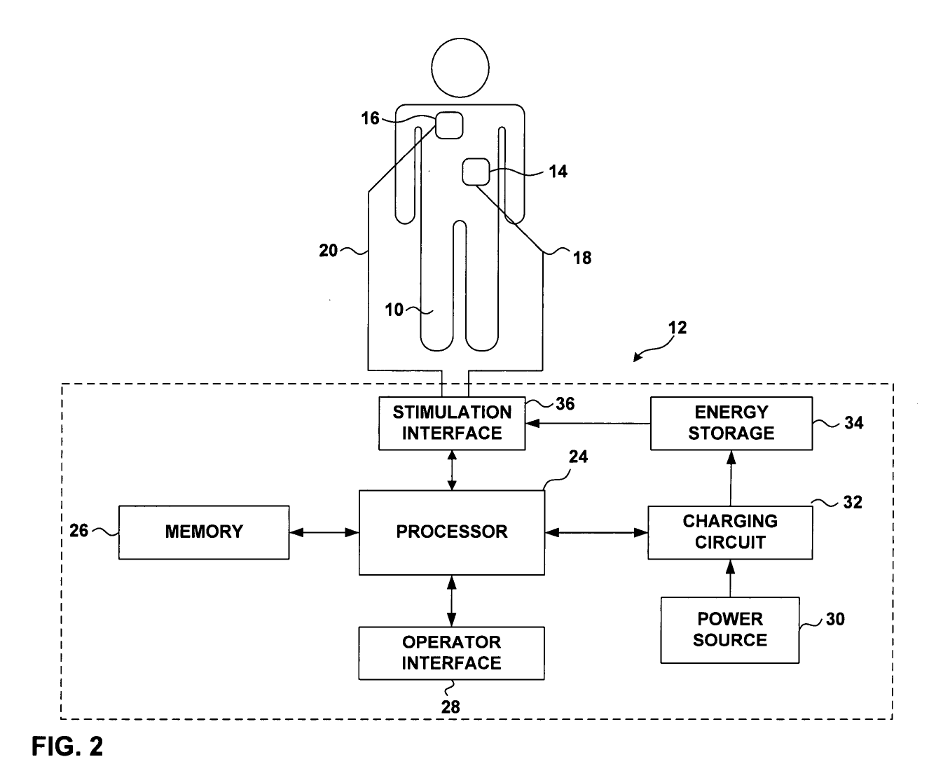

FIG. 2 is a block diagram illustrating exemplary components of external defibrillator 12 of FIG. 1 in further detail. As shown in FIG. 2, external defibrillator 12 may include a variety of components such as a processor 24, a memory 26, an operator interface 28, a power source 30, a charging circuit 32, an energy storage unit 34 and a stimulation interface 36.

In general, processor 24 controls the operations of external defibrillator 12 to deliver defibrillation pulses to patient 10 and monitor a condition of patient 10. Processor 24 may, for example, take the form of a microprocessor, microcontroller, an application specific integrated circuit (ASIC), field programmable logic array (FPGA), or other equivalent discrete or integrated logic circuitry. Memory 26 is accessible by processor 24, and may include program instructions that cause processor 24 to perform the functions attributed to processor 24 herein. Memory 26 may take the form of random access memory (RAM) or read-only memory (ROM), and contain program instructions that cause processor 24 to monitor the condition of patient 10, deliver defibrillation pulses to patient 10, and generate medical event information during the treatment of patient 10.

Electrodes 14 and 16 are coupled to external defibrillator 12 via leads 18 and 20 as well as stimulation interface 36. In a typical application, stimulation interface 36 includes one or more receptacles to receive leads 18 and 20. Stimulation interface 36 may include a switch (not shown) that, when activated, couples an energy storage unit 34 to leads 18 and 20. Energy storage unit 34 stores the energy to be delivered to patient 10 in the form of a defibrillation pulse. The switch may be of conventional design and may be formed, for example, by electrically operated relays that couple energy storage unit 34 to one or both of leads 18 and 20. Alternatively, the switch may comprise an arrangement of solid-state devices such as silicon-controlled rectifiers or insulated gate bipolar transistors.

Energy storage unit 34 includes components, such as one or more capacitors, that store the energy to be delivered to patient 10 via electrodes 14 and 16. Before a defibrillation pulse may be delivered to patient 10, energy storage unit 34 must be charged. Processor 24 directs charging circuit 32 to charge energy storage unit 34 to a high voltage level. Charging circuit 32 comprises, for example, a flyback charger that transfers energy from a power source 30 to energy storage unit 34. Power source 30 may comprise, for example, batteries and/or an adapter to an exterior power source such as an electrical outlet. In addition to supplying energy to charging circuit 32 and energy storage device 34, power source 30 also supplies power to other components of defibrillator 12 such as processor 24.

In operation, processor 24 of external defibrillator 12 receives an energy level for defibrillation selected by an operator of defibrillator 12. Processor 24 may receive the selection made by the operator via operator interface 28. Operator interface 28 may include input devices, such as a keypad and various buttons or dials, and output devices, such as various indicator lights, display monitors, speakers or other audible indicators. In some embodiments, the components illustrated in FIG. 2 may alternatively be arranged to form an automated external defibrillator (AED). Where the defibrillator is an AED, processor 24 may select an energy level from a preprogrammed progression of energy levels stored in memory 26 based on the number of defibrillation pulses already delivered to patient 10.

When the energy stored in energy storage unit 34 reaches the desired energy level, processor 24 controls operator interface 28 to provide an indication, such as an indicator light or a voice prompt, to the defibrillator operator indicating that defibrillator 12 is ready to deliver a defibrillation pulse to patient 10. The defibrillation pulse may be delivered manually or automatically. Where the defibrillation pulse is delivered manually, the external defibrillator operator may direct processor 24 to deliver the defibrillation pulse via operator interface 28, e.g., by pressing a button. In either case, processor 24 activates the switch to electrically connect energy storage unit 34 to leads 18 and 20, and thereby deliver the defibrillation pulse to patient 10 via electrodes 14 and 16.

External defibrillator 12 may further monitor electrical activity of the heart of patient 10 via electrodes 14 and 16, or via a set of dedicated sensing electrodes (not shown). For example, processor 24 may determine whether the heart of patient 10 is fibrillating based upon the sensed electrical activity in order to determine whether a defibrillation pulse should be delivered to patient 10. Where a defibrillation pulse has already been delivered, processor 24 may evaluate the efficacy of the delivered defibrillation pulse by determining if the heart is still fibrillating in order to determine whether an additional defibrillation pulse is warranted. Processor 24 may automatically deliver defibrillation pulses based on these determinations, or may advise the defibrillator operator of these determinations via operator interface 28.

Processor 24 drives display monitors 22 to display one or more measured patient parameters or operational status parameters to the operator via operator interface 28. Processor 24 may, for example, display an electrocardiogram (ECG) based on the sensed electrical activity, a heart rate or other patient parameters to the operator via operator interface 28. In accordance with the invention, operator interface 28 includes multiple display monitors 22 that display the patient parameters to the operator of external defibrillator 12. Display monitors 22 are arranged to allow the operator of external defibrillator 12 to continuously view patient parameters for constant monitoring of the condition of patient 10. For example, display monitors 22 may be arranged to allow the operator of external defibrillator 12 to view patient parameters while a cover of external defibrillator 12 is closed or during transportation of patient 10 with external defibrillator 12. For example, a major surface of a first display monitor is located on the same side of external defibrillator 12 as a handle, thus the operator may view the first display monitor while carrying external defibrillator 12 by the handle. A second display monitor may be located on a side adjacent the handle, such that the second display monitor may be viewed when external defibrillator 12 is placed on a surface, such as an ambulance shelf or the ground, but is not visible while the operator carries defibrillator 12 by its handle.

The operator may interact with processor 24 via operator interface 28 to identify which of the measured patient parameters and defibrillator status features to display. For example, the operator may interact with defibrillator 12 via an input medium, such as a keypad, and identify a subset of patient parameters to display on each of display monitors 22. The defibrillator operator may configure each of display monitors 22 of external defibrillator 12 to display the same patient parameters, a portion of the same patient parameters or completely different patient parameters.

Processor 24 may store in memory an indication of the time of delivery of each defibrillation pulse delivered to patient 10 as medical event information for patient 10 within memory 26. Processor 24 may also store the energy level of each pulse and other characteristics of each pulse, such as the width, amplitude, or shape, as medical event information for patient 10. Processor 24 may also store a digital representation of the ECG, heart rate and other measured data as patient parameters of patient 10. In this manner, processor 24 may obtain and store a record of patient diagnostic parameters, as well as operational status parameters, such as actions taken, observed patient responses, and other significant events during the course of the incident at the defibrillation scene. This information may take a variety of forms.

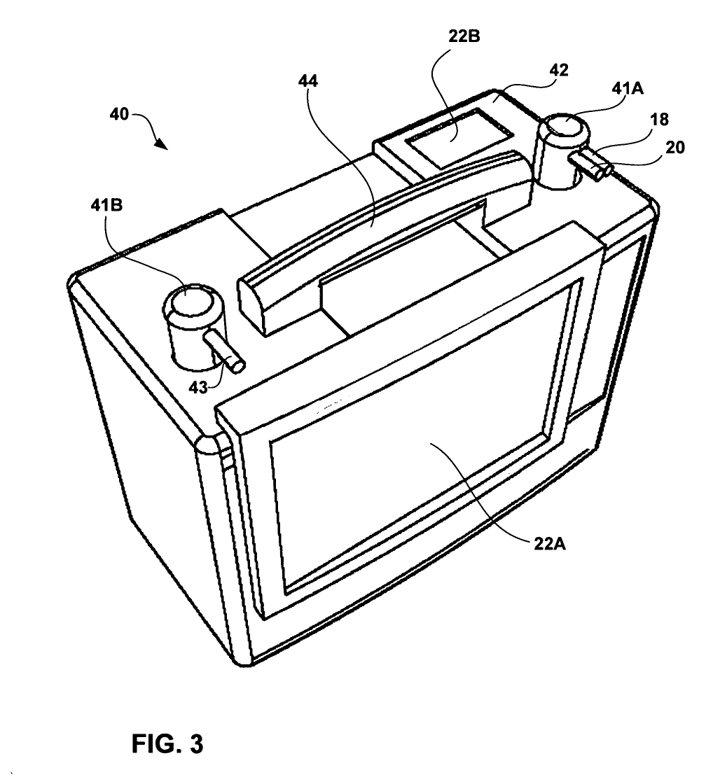

FIG. 3 is a schematic diagram illustrating an exemplary external defibrillator 40 that includes multiple display monitors. In particular, external defibrillator 40 includes display monitors 22A and 22B (“display monitors 22”) that allow an operator of external defibrillator 40 to continuously view patient parameters for constant monitoring of the condition of patient 10.

External defibrillator 40 includes receptacles 41A and 41B (“receptacles 41”). Receptacle 41A receives leads 18 and 20, while receptacle 41B receives a lead 42 that is coupled to a set of dedicated sensing electrodes, such as a set of ten electrodes for twelve-lead ECG collection. In addition, external defibrillator 40 includes a housing 42. Housing 42 may, for example, be constructed of plastic or other non-conductive material.

Display monitor 22B is visible at times when display monitor 22A would not be visible, such when defibrillator 40 is carried by a handle 44. In the illustrated example, display monitor 22A of external defibrillator 40 is located on first or “front” surface of housing 42, which is not able to be viewed by an operator while the operator carries external defibrillator 40 by handle 44, while display monitor 22B is located on the same surface of housing 42 that handle 44 is located.

In other words, a major surface of display monitor 22A is arranged in a first plane and a major surface of display monitor 22B is arranged in a second plane. The first and second planes corresponding to the major surfaces of display monitor 22A and 22B may be substantially perpendicular to one another, as illustrated in the example of FIG. 3. However, the invention is not limited to the illustrated perpendicular orientation of display monitors 22A and 22B. Display monitors 22A and 22B may be arranged such that they are oriented with respect to each other in any way, including display monitor 22A being arranged to be oriented substantially parallel to the plane in which display monitor 22B is arranged.

Display monitors 22A and 22B may each display the same patient parameters to the operator. In other words, display monitor 22B may be redundant. For example, display monitor 22B may display identical patient parameters as display monitor 22A in order for the operator of external defibrillator 40 to be able to continuously monitor patient 10. Alternatively, display monitor 22B may display only a subset of the patient parameters that display monitor 22A displays. For example, display monitor 22A may display a heart rate, an ECG based on the sensed electrical activity, patient information such as age, weight, and the like, as well as additional patient parameters, while display monitor 22B displays a subset of the patient parameters displayed by display monitor 22A, e.g., the heart rate and ECG of patient 10.

Display monitor 22B may only display a subset of the patient parameters displayed by display monitor 22A due to the size of display monitor 22B. For instance, in order to incorporate display monitor 22B within the surface of external defibrillator 40 where handle 48 is located, display monitor 22B may need to be a smaller size. Display monitor 22A, display monitor 22B, or both may further display defibrillator status features. For example, either of the display monitors may show a battery icon, a charging icon, a charged icon, or similar operation status feature of external defibrillator 40.

Display monitor 22A may be a “main” display, i.e., used during regular operation of external defibrillator 12, while display monitor 22B may be a secondary display, e.g., only used to monitor the condition of patient 10 during transport. Display monitor 22B may access all the functional components of external defibrillator 40 in the same manner as display monitor 22A. For example, display monitor 22B may function as a touch screen, thereby acting as an input medium via which the operator interacts with external defibrillator 40.

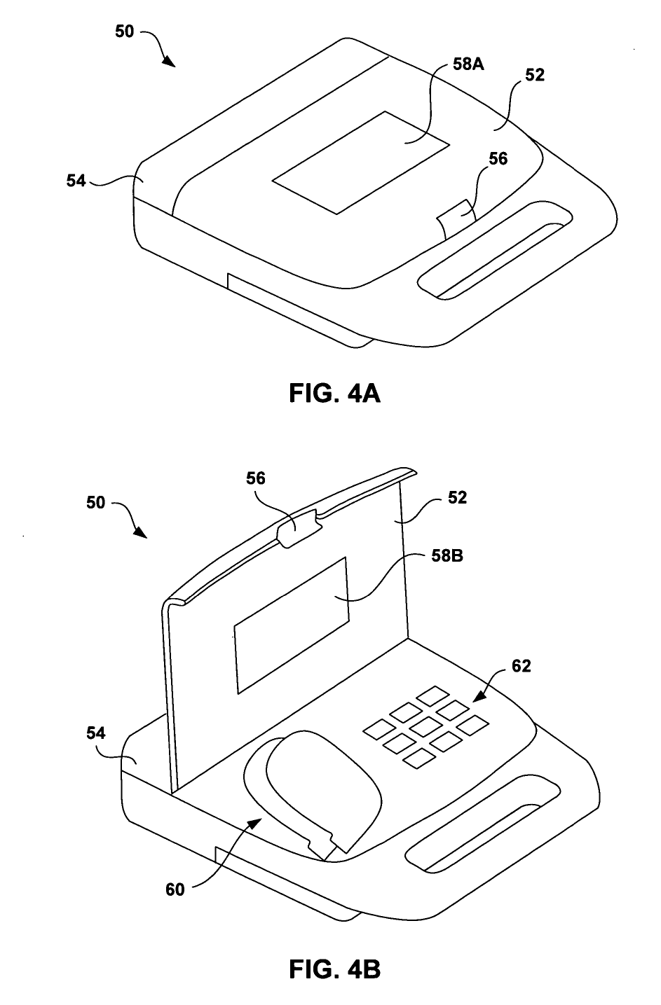

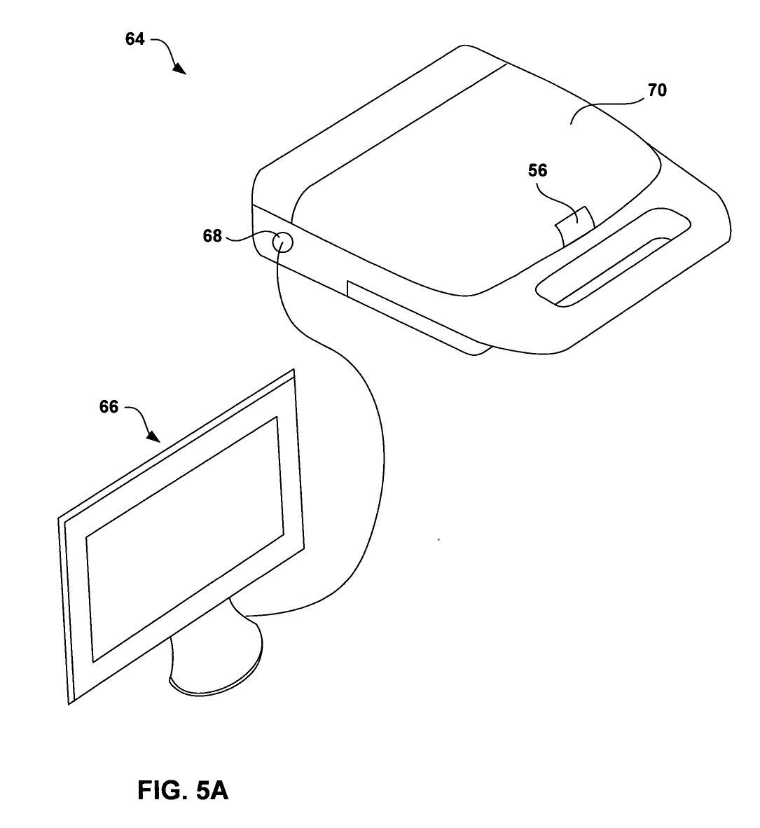

FIGS. 4A and 4B are schematic diagrams illustrating another exemplary external defibrillator 50 that includes multiple display monitors. In particular, FIG. 4A illustrates external defibrillator 50 with a cover 52 of external defibrillator 50 in a closed position and FIG. 4B illustrates external defibrillator 50 with cover 52 in an open position.

External defibrillator 50 includes a housing 54 and cover 52 is attached to housing 54. Cover 52 pivots around hinges (not shown) to open and close. In addition, cover 52 of external defibrillator 50 may include a locking mechanism 56 to secure cover 52 in a closed position during transportation or storage. For example, when cover 52 is closed, locking mechanism 56 is received by a portion of housing 54 and secures cover 52 in a closed position. In order to open cover 52, for instance, locking mechanism 56 may be pushed inward and cover 52 may be pulled open.

Cover 52 includes display monitors 58A and 58B (“display monitors 58”). Display monitor 58A is located on an outer surface of cover 52, i.e., the surface of cover 52 that is visible when cover 52 is in the closed position, as illustrated in FIG. 4A. Display monitor 58A may be used to display patient parameters to an operator of defibrillator 50 while cover 52 is in the closed position. Display monitor 58B is located on the inner surface of cover 52, i.e., on the surface of cover 52 that is not visible when cover 52 is in the closed position, as illustrated in FIG. 4B. In this manner, the main surfaces of each of display monitors 58A and 58B are substantially parallel. Display monitors 58 may comprise Cathode Ray Tube (CRT) display monitors, Light Emitting Diode (LED) monitors, Liquid Crystal Display (LCD) monitors, or plasma display monitors.

Display monitor 58A may be used to display patient parameters to an operator of external defibrillator 50 while cover 52 is in the closed position, e.g., during transportation of defibrillator 50. In one embodiment, display monitor 58A displays patient parameters while cover 52 is in a closed position, but as soon as cover 52 is opened display monitor 58A is turned off and display monitor 58B begins to display patient parameters. Display monitor 58A turns back on and display monitor 58B shuts off when the cover is closed. Alternatively, display monitor 58A may be on while cover 52 is in the closed or open position. In this manner, display monitor 58A and display monitor 58B display patient parameters to the operator concurrently. This way, the operator may be on either side of external defibrillator 50 and view patient parameters.

As described above, display monitors 58A and 58B may display the same patient parameters, a portion of the same patient parameters, or completely different patient parameters. For example, display monitor 58A may display only a subset of the patient parameters that display monitor 58B displays. The patient parameters may include, for example, vital statistics, ECG waveforms, diagnoses, and the like. Display monitor 58A, display monitor 58B, or both may further display defibrillator status features. For example, either of the display monitors may show a battery icon, a charging icon, a charged icon, or similar operation status feature of external defibrillator 50.

When cover 52 is in the open position, the operator may access electrodes 60, which may be either hand-held electrode paddles or adhesive electrode pads placed on the skin of patient 10. Additionally, opening cover 52 further exposes an input medium via which the operator interacts with external defibrillator 50. In the example illustrated in FIG. 4B, the input medium of external defibrillator 50 comprises a keypad 62. Keypad 62 may take the form of an alphanumeric keypad or a reduced set of keys associated with particular functions. In some embodiments, one or both of display monitors 58 may be a touch screen display, and the operator may interact with external defibrillator 50 via display monitors 58. The operator may also interact with external defibrillator 50 using a peripheral pointing device, such as a stylus or mouse. The operator may, for example, interact with external defibrillator 50 via keypad 62 to identify which of the measured patient parameters and defibrillator status features to display via each of display monitors 58.

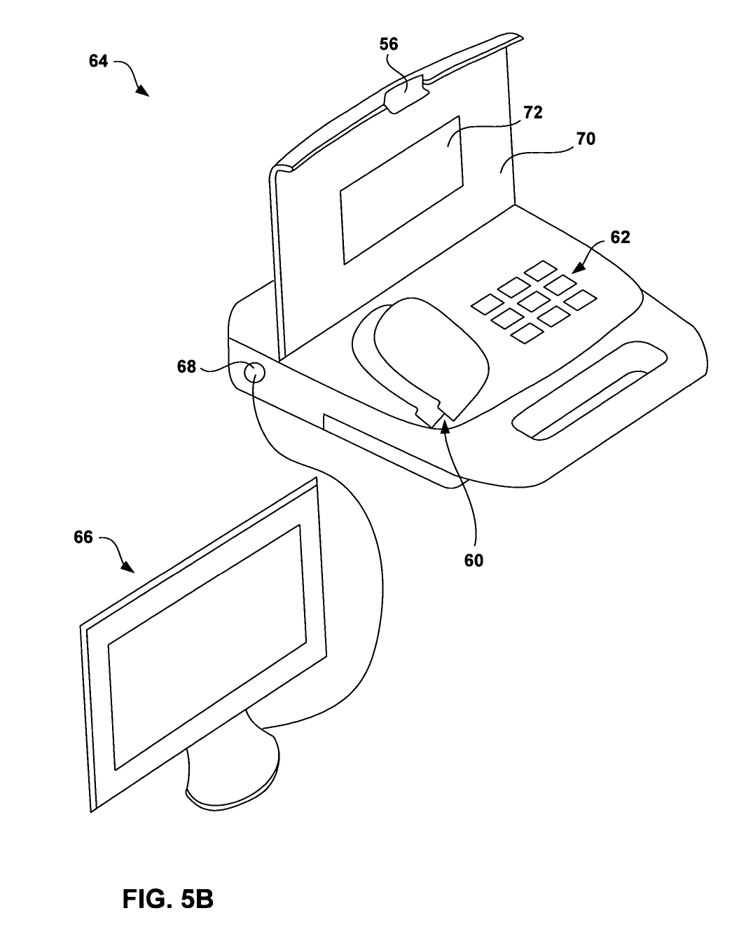

FIGS. 5A and 5B are schematic diagrams illustrating yet another exemplary external defibrillator 64 that includes multiple display monitors. External defibrillator 64 conforms substantially to external defibrillator 50 of FIGS. 4A and 4B, but one of the display monitors is detached from external defibrillator 64. In particular, external defibrillator 64 is coupled to detached display monitor 66 via a port 68 instead of both of the display monitors being integrated within cover 52 as illustrated in external defibrillator 50 of FIGS. 4A and 4B. Port 68 may be a wired port or a wireless port. In one example, port 68 may be a wired port, and receive a plug from detached display monitor 66 to electrically couple external defibrillator 64 to detached display monitor 66. In another example, port 68 may be a wireless port, such as an infrared (IR) or radio frequency (RF) port, and wirelessly couple detached display monitor 66 to external defibrillator 64 using wireless telemetry or other wireless communication techniques.

Detached display monitor 66 may display patient parameters to an operator of external defibrillator 64 while a cover 70 is in the closed position, as illustrated in FIG. 5A. In one embodiment, detached display monitor 66 may turn off when cover 70 is opened, as illustrated in FIG. 5B, and a display monitor 72 may display the patient parameters to the operator, e.g., display monitors 66 and 72 successively display patient parameters. Alternatively, detached display monitor 66 may continue to display patient parameters while cover 70 is in the open position. In other words, detached display monitor 66 and display monitor 72 display patient parameters concurrently. In this manner, external defibrillator 64 continuously displays patient parameters to the operator in order to constantly monitor the condition of patient 10.

Although illustrated in FIGS. 5A and 5B as a freestanding monitor, a display monitor 66 that is detached from defibrillator 64 may take a variety forms according to various embodiments of the invention. For example, in some embodiments display monitor 66 comprises a display that is a component of a portable computing device, such as a display of a laptop of handheld computer, a personal digital assistant (PDA), or a cellular telephone. In some embodiments, display monitor 66 may be worn by the operator of defibrillator 64. For example, a detached display monitor 66 may be sized such that it can be secured to a wrist of the operator by a strap, or may be a component of a wristwatch.

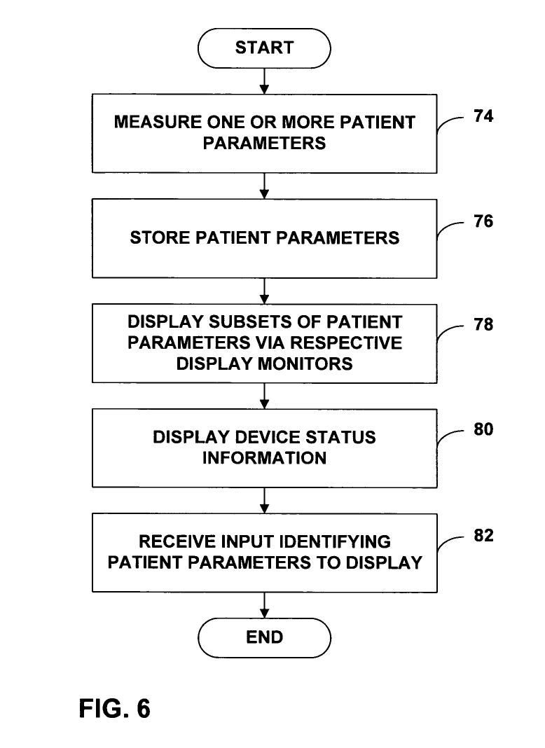

FIG. 6 is a flow diagram illustrating exemplary operation of an external defibrillator 12 with multiple display monitors displaying patient parameters to an operator in accordance with the invention. Initially, external defibrillator 12 measures one or more patient parameters of patient 10 (74). External defibrillator 12 may, for example, measure patient parameters via electrodes 14 and 16 that are placed on the skin of patient 12. Patient parameters that external defibrillator 12 may measure include heart rate of patient 10, an ECG waveform of patient 10, or other vital or non-vital parameters of patient 10.

External defibrillator 12 stores the measured patient parameters in memory 26 (76) and displays at least a portion of the patient parameters to a defibrillator operator via multiple display monitors (78). In accordance with the invention, the display monitors are arranged to allow the external defibrillator operator to continuously view patient parameters. In one example, the display monitors are arranged such that the operator can view a major surface of one of display monitors while carrying external defibrillator 12 by a handle to allow the operator to view patient parameters during transportation of external defibrillator 12 and patient 10.

The other display monitor may be located on a side of external defibrillator 12 not facing the external defibrillator operator while the operator is carrying defibrillator 12 by a handle, or may be enclosed by a cover. In another example, one of the display monitors may be located on the outside surface of the cover of external defibrillator 12 and another one of display monitors 22 is located on an inside surface of the cover of external defibrillator 12, i.e., the display monitors are located on opposite sides of the cover, to allow the external defibrillator operator to view patient parameters while the cover of external defibrillator 12 is closed. In a further embodiment, one of the display monitors may be detached. In addition, the first display monitor, the second display monitor, or both may display external defibrillator status features (80).

External defibrillator 12 may receive input from the operator identifying which of the patient parameters and defibrillator status features to display (82). For example, the operator may interact with external defibrillator 12 via an input medium, such as a keypad, and identify a subset of patient parameters to display on each of the display monitors. As described above, the external defibrillator operator may configure each of the display monitors of external defibrillator 12 to display the same patient parameters, a portion of the same patient parameters or completely different patient parameters.

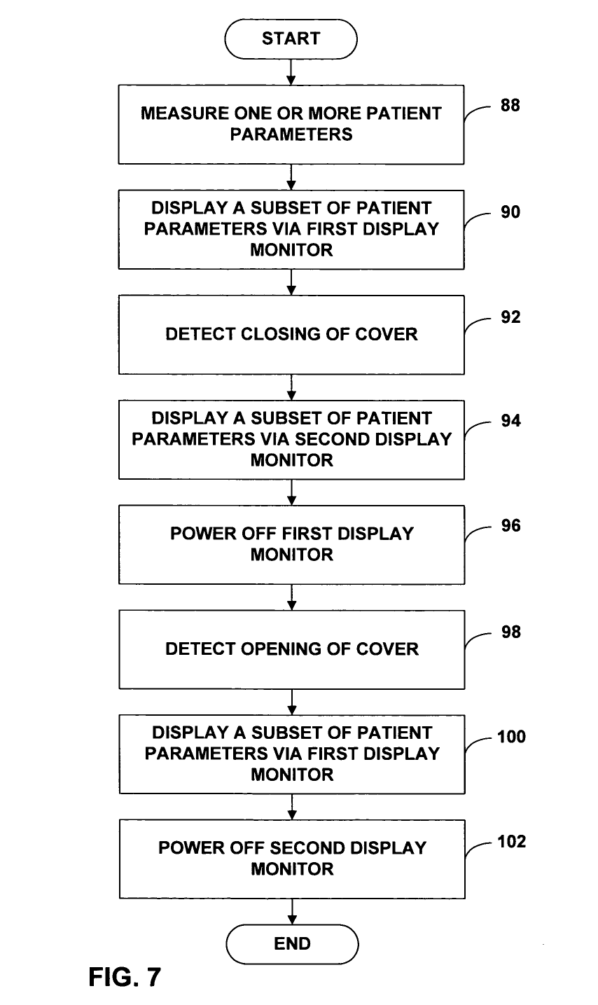

FIG. 7 is a flow diagram illustrating exemplary operation of an external defibrillator 12 with multiple display monitors switching display monitors upon opening and closing of a cover of external defibrillator 12. Initially, external defibrillator 12 measures one or more patient parameters of patient 10 (88). External defibrillator 12 may, for example, measure patient parameters via electrodes 14 and 16 that are placed on the skin of patient 12. External defibrillator 12 displays at least a subset of the measured patient parameters via a first display monitor (90). The first display monitor may be a “main” display monitor that is used to display patient parameters during regular operation. For example, the first display monitor may be a display monitor integrated within a cover of external defibrillator 12, and that is viewable by the operator of external defibrillator 12 when the cover is in the open position.

In response to the operator closing the cover, external defibrillator 12 detects the closure of the cover (92). The operator may, for example, close the cover of external defibrillator 12 in order to transport the patient as well as external defibrillator 12 from the current location to an ambulance. Upon detecting closure of the cover, external defibrillator 12 displays a second subset of patient parameters to the operator via a second display monitor (94) and powers off the first display monitor (96). In this manner, the operator may view patient parameters continuously in order to constantly monitor the condition of patient 10.

The second display monitor may, for example, be located on the outer surface of the cover. In this manner, the cover has a display screen on both sides in order to allow the operator to view the patient parameters continuously. In another example, the second display monitor may be integrated within a housing of external defibrillator 12 and, more particularly, on a side of external defibrillator 12 where a handle is located. In some embodiments the second display monitor may be integrated within the handle itself. In this manner, the operator may easily view the patient parameters during transportation of patient 10 and external defibrillator 12.

The first and second display monitors display the same patient parameters, a portion of the same patient parameters or completely different patient parameters. In addition, the first display monitor, the second display monitor, or both may display defibrillator status features. In one example, the first display monitor, e.g., the “main” display monitor, may display a heart rate, an ECG based on the sensed electrical activity, patient information such as age, weight, and the like, as well as a battery icon and a charging icon. The second display monitor may only display a subset of the patient parameters displayed by main display monitor, e.g., the heart rate, ECG of patient 10, or other vital signals.

In response to the operator opening the cover, external defibrillator 12 detects the opening of the cover (98). The operator may, for example, open the cover of external defibrillator 12 upon placement of patient 10 in the ambulance. Upon detecting opening of the cover, external defibrillator 12 displays the first subset of patient parameters to the operator via the first display monitor (100), i.e., the main display monitor. External defibrillator 12 may also power of the second display monitor upon displaying the patient parameters via the main display monitor (102).

Various embodiments of the invention have been described. These and other embodiments are within the scope of the following claims.

1. An emergency medical device comprising:
at least one sensor to measure one or more patient parameters;
a first display monitor to display at least a first subset of the patient parameters to an operator; and
a second display monitor to display at least a second subset of the patient parameters to the operator.

2. The emergency medical device of claim 1, in which a major surface of the first display monitor is arranged in a first plane and a major surface of the second display monitor is arranged in a second plane, and the first plane and the second plane are substantially perpendicular to one another.

3. The emergency medical device of claim 1, in which a major surface of the first display monitor is arranged in a first plane and a major surface of the second display monitor is arranged in a second plane, and the first plane and the second plane are substantially parallel to one another.

4. The emergency medical device of claim 1, further comprising a housing and a cover attached to the housing, the first display monitor and the second display monitor being located within the cover, in which the first display monitor is located on one side of the cover and the second display monitor is located on the opposite side of the cover.

5. The emergency medical device of claim 4, in which the first display monitor displays the first subset of patient parameters when the cover is closed.

6. The emergency medical device of claim 5, in which the first display monitor is turned off when the cover is opened.

7. The emergency medical device of claim 4, in which the second display monitor displays the second subset of patient parameters when the cover is open.

8. The emergency medical device of claim 7, in which the second display monitor is turned off when the cover is closed.

9. The emergency medical device of claim 4, in which the first display monitor displays the first subset of patient parameters and the second display monitor displays the second subset of patient parameters when the cover is open.

10. The emergency medical device of claim 1, further comprising a handle, and in which the first display monitor is located on a side of the device on which the handle is located and the second display monitor is located on a side of the device adjacent to the handle.

11. The emergency medical device of claim 1, in which the first display monitor displays the first subset of patient parameters and the second display monitor displays the second subset patient parameters concurrently.

12. The emergency medical device of claim 1, in which the first subset of patient parameters and the second subset of patient parameters include at least some of the same patient parameters.

13. The emergency medical device of claim 1, in which the first subset of patient parameters and the second subset of patient parameters include at least one different patient parameter.

14. The emergency medical device of claim 1, in which at least one of the first display monitor and the second display monitor displays emergency medical device status information in addition to the subset of patient parameters.

15. The emergency medical device of claim 1, further comprising a port that electrically couples to the first display monitor.

16. The emergency medical device of claim 15, in which the port comprises one of a wired port and a wireless port, and the first display monitor is detached from the emergency medical device.

17. The emergency medical device of claim 16, in which the first display monitor comprises a display that is a component of one of a laptop computer, a handheld computer, a personal digital assistant, a cellular telephone, and a wristwatch.

18. The emergency medical device of claim 1, further comprising a memory to store the measured patient parameters, in which the emergency medical device displays the stored patient parameters via at least one of the first display monitor and second display monitor.

19. The emergency medical device of claim 1, in which the first display monitor and the second display monitor comprise one of a Liquid Crystal Display (LCD) monitor, Light-Emitting Diode (LED) display monitor, and a plasma display monitor.

20. The emergency medical device of claim 1, in which the device comprises an external defibrillator.

21. The emergency medical device of claim 1, in which the device comprises one of an external therapeutic emergency medical device and an external diagnostic emergency medical device.

22. A method comprising:
measuring one or more patient parameters;
displaying a first subset of the measured patient parameters to an operator via a first display monitor of an emergency medical device; and
displaying a second subset of the measured patient parameters to the operator via a second display monitor of the emergency medical device.

23. The method of claim 22, in which a major surface of the first display monitor is arranged in a first plane and a major surface of the second display monitor is arranged in a second plane, and the first plane and the second plane are substantially perpendicular to one another.

24. The method of claim 22, in which a major surface of the first display monitor is arranged in a first plane and a major surface of the second display monitor is arranged in a second plane, and the first plane and the second plane are substantially parallel to one another.

25. The method of claim 22, in which the first display monitor and the second display monitor are located within a cover that is attached to a housing of the emergency medical device, the first display monitor being located on one side of the cover and the second display monitor located on the opposite side of the cover.

26. The method of claim 25, in which displaying the first subset of the measured patient parameters to the operator via the first display monitor of the emergency medical device comprises displaying the first subset of the measured patient parameters to the operator via the first display monitor of the emergency medical device when the cover is closed.

27. The method of claim 26, further comprising powering off the first display monitor upon detecting opening of the cover.

28. The method of claim 25, in which displaying the second subset of the measured patient parameters to the operator via the second display monitor of the emergency medical device comprises displaying the second subset of the measured patient parameters to the operator via the second display monitor of the emergency medical device upon detecting opening of the cover.

29. The method of claim 28, further comprising powering off the second display monitor upon detecting closure of the cover.

30. The method of claim 22, in which the first display monitor is located on a side on which a handle is located and the second display monitor is located on a side adjacent to the handle.

31. The method of claim 22, in which the first subset of patient parameters and the second subset of patient parameters include at least some of the same patient parameters.

32. The method of claim 22, in which the first subset of patient parameters and the second subset of patient parameters include at least one different patient parameter.

33. The method of claim 22, further comprising receiving input from the operator identifying at least one of the subsets of patient parameters to display.

34. The method of claim 22, further comprising displaying emergency medical device status information to the operator via at least one of the first display monitor and the second display monitor.

35. The method of claim 22, further comprising:
storing measured patient parameters; and
displaying the stored patient parameters to the operator via at least one of the first display monitor and the second display monitor.

36. The method of claim 22, in which the emergency medical device comprises an external defibrillator.

37. The method of claim 22, in which the emergency medical device comprises one of an external therapeutic emergency medical device and an external diagnostic emergency medical device.

38. The method of claim 22, in which the first display monitor comprises a display that is a component of one of a laptop computer, a handheld computer, a personal digital assistant, a cellular telephone, and a wristwatch.
